(19) United States
(12) Patent Application Publication
(10) Pub. No.: US 2024/0131274 A1
(43) Pub. Date: Apr. 25, 2024

(54) DEVICE FOR ADDRESSING NEEDLESTICK RELATED INJURIES IN INJECTION PRACTICES

(71) Applicants: Kyle BAYLOUS, Shoreham, NY (US); Daniel MELTZER, Melville, NY (US); Ryan TAM, Staten Island, NY (US); Christopher HELENEK, Lake Grove, NY (US); Jesse LOBENHOFER, Stony Brook, NY (US)

(72) Inventors: Kyle BAYLOUS, Shoreham, NY (US); Daniel MELTZER, Melville, NY (US); Ryan TAM, Staten Island, NY (US); Christopher HELENEK, Lake Grove, NY (US); Jesse LOBENHOFER, Stony Brook, NY (US)

(21) Appl. No.: 18/278,313

(22) PCT Filed: Feb. 23, 2022
(86) PCT No.: PCT/US2022/017410

§ 371 Date: Aug. 21, 2023

Related U.S. Application Data
(60) Provisional application No. 63/152,395, filed on Feb. 23, 2021.

(51) Int. Cl. A61M 5/32

(52) U.S. Cl. CPC A61M 5/3257; A61M 2209/088

(57) ABSTRACT

A device that includes a sheath, wherein the sheath includes an opening configured to receive a container; a connector conduit, a first end of the connector conduit operably connected to the sheath, a connector rod, wherein a second end of the connector conduit, the second end of the connector conduit opposite the first end of the connector conduit, is operably connected to a first end of the connector rod; and a clamp, wherein a second end of the connector rod, the second end of the connector rod opposite the first end of the connector rod, is operably connected to the clamp, and wherein the clamp is configured to extend at least partially around the perimeter of a syringe barrel.

### CROSS REFERENCE TO RELATED APPLICATION

This application claims the benefit of priority from U.S. Provisional Application No. 63/152,395 filed Feb. 23, 2021, the entire contents of which are incorporated herein by reference.

### BACKGROUND OF THE DISCLOSURE

The present disclosure relates to a needlestick prevention device for use with syringes to administer injections, while providing vial stabilization for the withdrawal phase of injection.

A needlestick injury generally occurs in a medical environment, particularly during use of a syringe, when the user accidentally sticks the needle into themself or another person. The occurrence of needlestick injuries should be reduced and/or prevented, as they can spread diseases and infections, while also causing pain for the person who is stuck.

Needlestick injuries may occur at any time of injection, including while filling the syringe with a drug substance from a vial, while administering the injection, and while disposing of the injection device. A common form of protection against an injection needle is a protective cap, which is removed to administer an injection. Protective caps are typically disposed of following the delivery of an injection, as the replacement of the cap is thought to be a significant cause of needlestick injuries. While this method does protect against needlestick injury before injection, protection is not provided against a needlestick injury during and after the injection. Needlestick injuries with injections can still occur.

Another technique for preventing needlestick injuries during the phases of an injection includes the use of a safety shield, which is mounted on the needle-bearing syringe. The safety shield provides protection against needlestick injury once the disposable protective cap is removed. The user pivots the shield away from the needle to administer the injection, and pivots the shield back towards the needle into a locked position to protect from needlestick injury after the injection. This method alone provides protection against a needlestick injury after an injection, and when used in combination with a protective cap, provides protection against a needlestick injury both before and after an injection. Unfortunately, protection is not provided against a needlestick injury during the injection.

Additionally, products exist termed “vial adaptors” that aid in the safe withdrawal of liquid from vials, but such devices are not intended to be used for needlestick prevention purposes, nor do these products provide the ability to administer an injection while in use.

In light of the above, there is a present need for a needlestick prevention device that protects the user, or another person, from needlestick injuries which may occur while filling the syringe with a substance from a vial, while administering the injection, and once the injection is completed and the injection device is disposed of.

Embodiments of the present disclosure provide devices and methods that address the above needs, and others.

### SUMMARY OF THE DISCLOSURE

The present disclosure is directed to a device that includes a sheath, wherein the sheath includes an opening configured to receive a container; a connector conduit, a first end of the connector conduit operably connected to the sheath, a connector rod, wherein a second end of the connector conduit, the second end of the connector conduit opposite the first end of the connector conduit, is operably connected to a first end of the connector rod; and a clamp, wherein a second end of the connector rod, the second end of the connector rod opposite the first end of the connector rod, is operably connected to the clamp, and wherein the clamp is configured to extend at least partially around the perimeter of a syringe barrel.

The present disclosure is also directed to a method of preparing an injection that includes: moving a vial into an opening of a device, the vial including a container, a vial cap and a liquid and/or aqueous solution, the device including: a sheath, wherein the sheath comprises an opening configured to receive a container; a connector conduit, a first end of the connector conduit operably connected to the sheath, a connector rod, wherein a second end of the connector conduit, the second end of the connector conduit opposite the first end of the connector conduit, is operably connected to a first end of the connector rod; and a clamp, wherein a second end of the connector rod, the second end of the connector rod opposite the first end of the connector rod, is operably connected to the clamp, and wherein the clamp is configured to extend at least partially around the perimeter of a syringe barrel of a syringe; inserting the syringe barrel into the clamp; inserting a needle of the syringe into the vial; withdrawing at least a portion of the liquid and/or aqueous solution through the needle, into the syringe barrel; removing the needle from the vial; and removing the vial from the opening of the device.

### BRIEF DESCRIPTION OF THE DRAWINGS

The present disclosure will be better understood by reference to the following drawings, which are provided as illustrative of certain embodiments of the subject application, and not meant to limit the scope of the present disclosure.

FIG. 1 shows a top side orthogonal view of a first embodiment the needlestick prevention device having a vial stabilizer.

FIG. 2 shows a bottom side orthogonal view of the device of FIG. 1.

FIG. 3 shows a front side orthogonal view of the device of FIG. 1.

FIG. 4 shows a rear side orthogonal view of the device of FIG. 1.

FIG. 5 shows a top perspective view of the device of FIG. 1.

FIG. 6 shows rear perspective view of the device of FIG. 1.

FIG. 7 shows a top side cross sectional view of the device of FIG. 1.

FIG. 8 shows a right side cross sectional view of the device of FIG. 1.

FIG. 9 shows a right perspective view of the first embodiment of the needlestick prevention device attached to a conventional syringe, with a vial loaded into the vial stabilizer.

FIG. 10 shows top perspective view of the assembled devices of FIG. 9.

FIG. 11 shows a top side cross sectional view of the assembled devices of FIG. 9.

FIG. 12 shows a right perspective view of the first embodiment of the needlestick prevention device attached to a conventional syringe during the injection.

FIG. 13 shows a right perspective view of the first embodiment of the needlestick prevention device attached to a conventional syringe with the syringe needle protected within the sheath.

FIG. 14 shows a top perspective view of a second embodiment of the device, including a stopper.

FIG. 15 shows a left perspective view of the device of FIG. 14.

FIG. 16 shows a top perspective view of the second embodiment of the device, with the stopper component visible, attached to a conventional syringe with a vial loaded into the vial stabilizer.

FIG. 17 shows a top side cross sectional view of the assembled devices of FIG. 16.

FIG. 18 shows a left perspective view of the second embodiment of the device with the stopper in place after being inserted into the slot. The device is attached to a conventional syringe and the needle is in contact with the stopper, with the vial stabilizer unloaded.

FIG. 19 shows a right cross sectional view of the device of FIG. 18.

FIG. 20 shows a left cross sectional view of the device of FIG. 18.

### DETAILED DESCRIPTION OF THE DISCLOSURE

The following detailed description of embodiments of the disclosure are made in reference to the accompanying figures. Explanation about related functions or constructions known in the art are omitted for the sake of clearness in understanding the concept of the invention to avoid obscuring the invention with unnecessary detail. Embodiments of the disclosure described herein provide.

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein.

As used herein, the term “substantially”, or “substantial”, is equally applicable when used in a negative connotation to refer to the complete or near complete lack of an action, characteristic, property, state, structure, item, or result. For example, a surface that is “substantially” flat would either completely flat, or so nearly flat that the effect would be the same as if it were completely flat.

As used herein terms such as “a”, “an” and “the” are not intended to refer to only a singular entity, but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element, and a second element, are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

The embodiments of the present disclosure can be used in conjunction with several presently produced elements, including needles, syringes, and vials currently. Although the term “vial” is referred to throughout, this “vial” includes any suitable container that can store a liquid and/or aqueous solution that is capable of being suctioned by a needle of any suitable size.

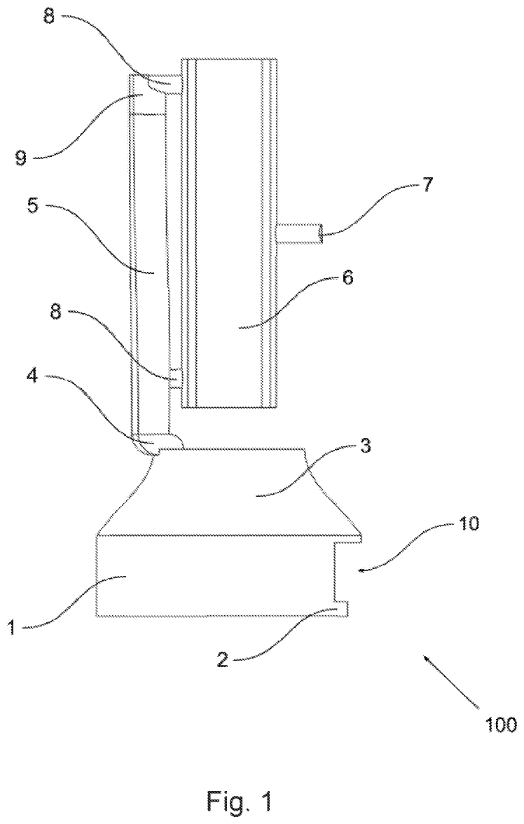

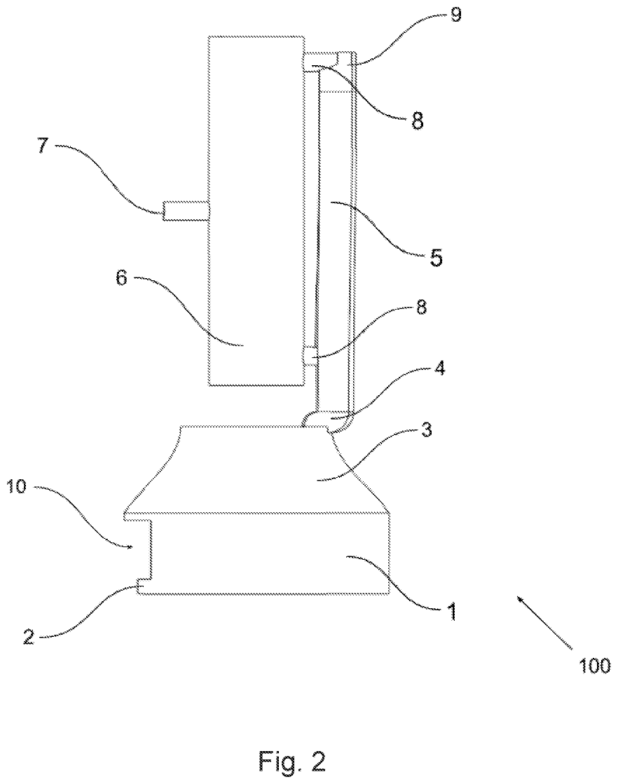

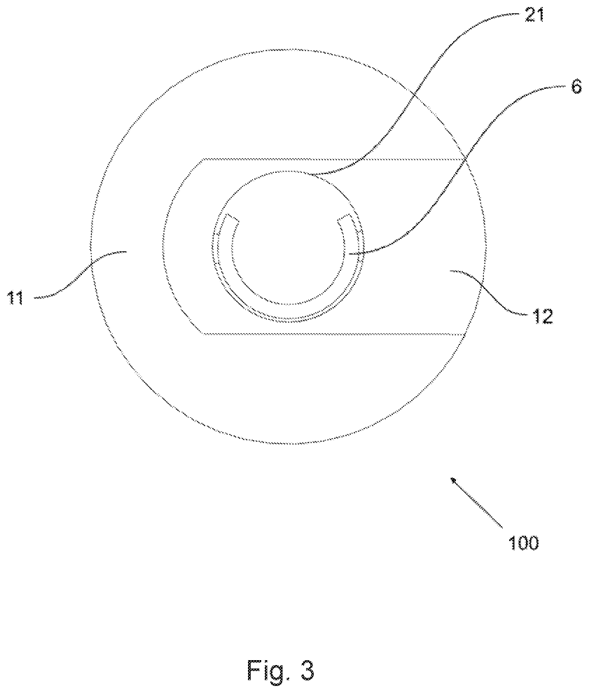

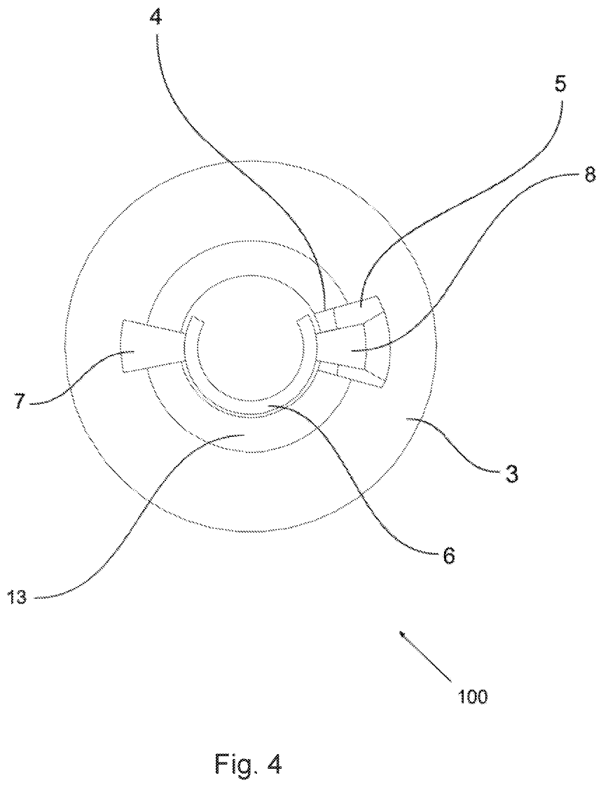

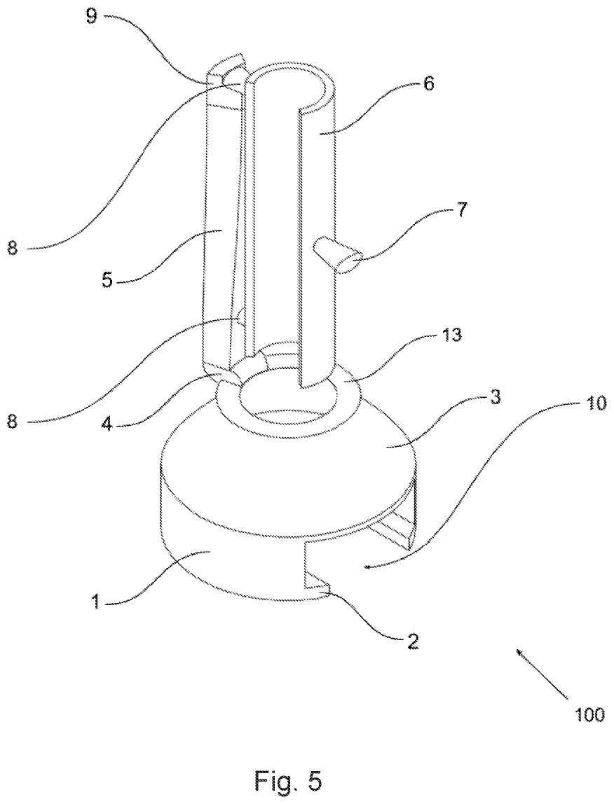

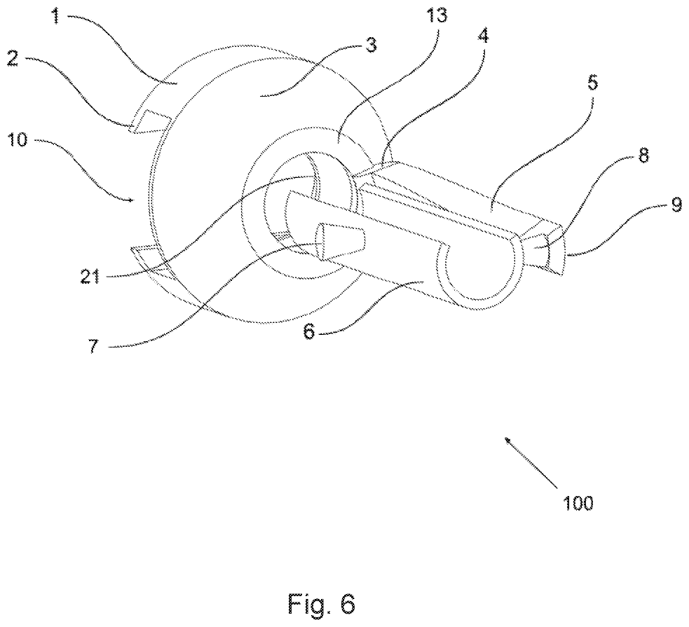

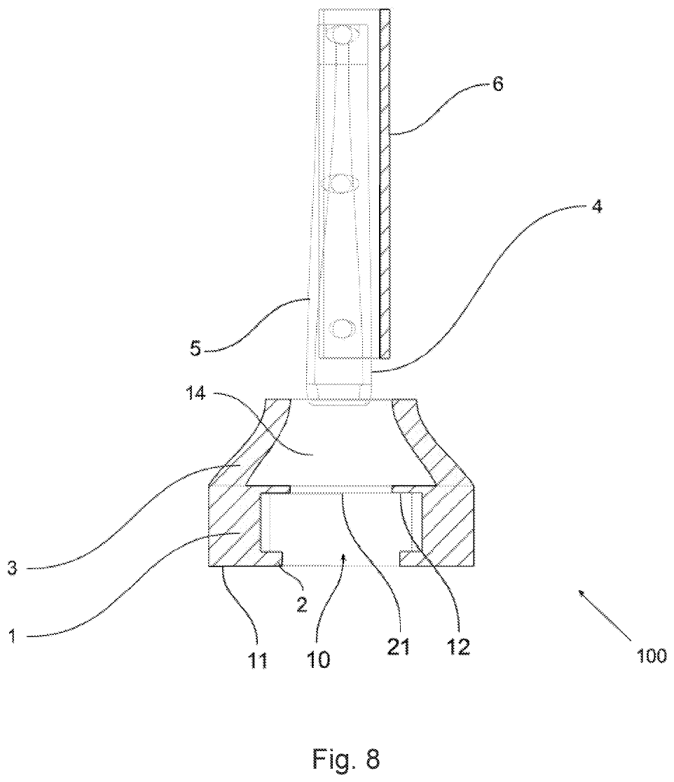

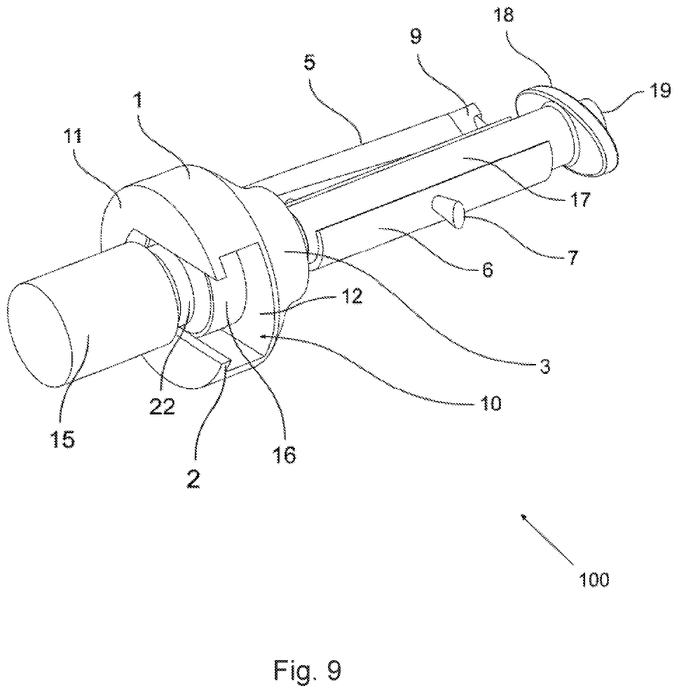

In FIGS. 1-13, directed to a first embodiment of a device 100, the same reference numbers refer to similar elements in each figure. Referring to FIG. 1, device 100 is configured to connect a syringe through the clamp 6, and a vial body 15 through a vial opening 10. The clamp 6 is configured to at least partially surround a syringe barrel 17 (as shown in FIG. 9), with a sheath 1 being between a needle 20 (operably and fluidly connected of the syringe barrel 17) and a user. While the device 100 contains a sheath 1 that is illustrated as substantially conical, the sheath 1 is not limited to a conical shape, and can adopt other shapes as well, including but not limited to a substantially rectangular shape, a substantially cylindrical shape, and a substantially spherical and/or round shape. The surface of the sheath 1 vertically lowest in FIGS. 1 and 2 can be a substantially flat front face 11 that is configured to contact a patient's skin during use of the device 100. However, in other embodiments, the front face 11 can be any suitable shape to contact a patient's skin, such as wholly or partially concave, wholly or partially convex, an erratic curvature, and/or have one or more shapes of extensions or bumps extending therefrom.

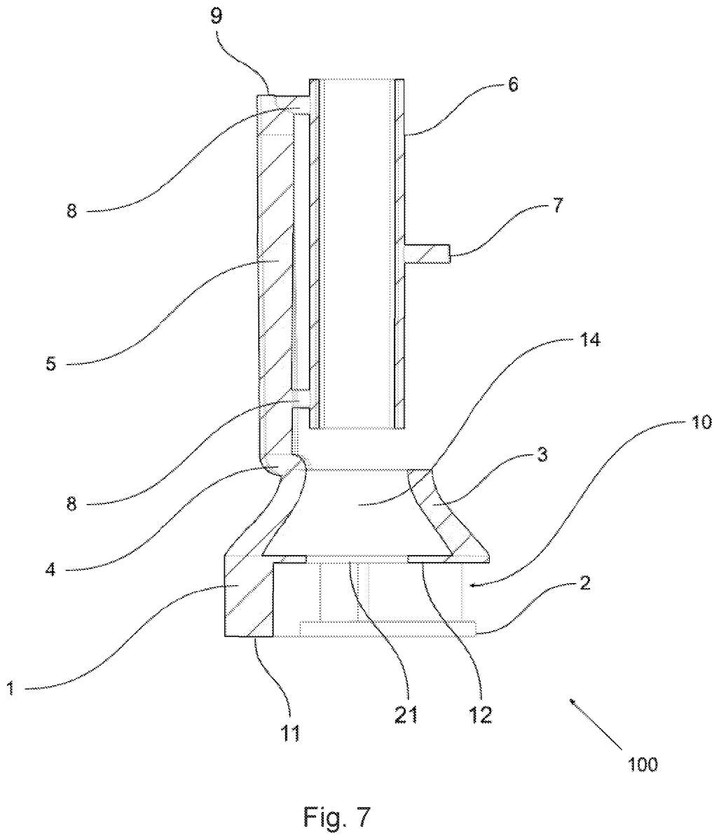

As seen in FIG. 9, the syringe barrel 17, the clamp 6, the vial body 15, in the vial opening 10, are connected through the device 100. The clamp 6 is connected via a connector post 8 (as seen in FIG. 7) to the rail connector 5. Although two connector posts 8 are shown in FIG. 7, in other embodiments, one, three or more connector posts 8 can extend between the clamp 6 and the rail connector 5. Each connector post 8 can be solid or hollow, and can have any suitable cross-sectional shape between the clamp 6 and the rail connector 5, including but not limited to a substantially rectangular shape, a substantially conical shape, a substantially cylindrical shape, etc. The rail connector 5 is connected to the sheath 1, the tapered neck 3, and the tapered backing 13 via the connector region 4. The sheath 1 contains the vial opening 10, thus the current device 100 allows these components to be connected.

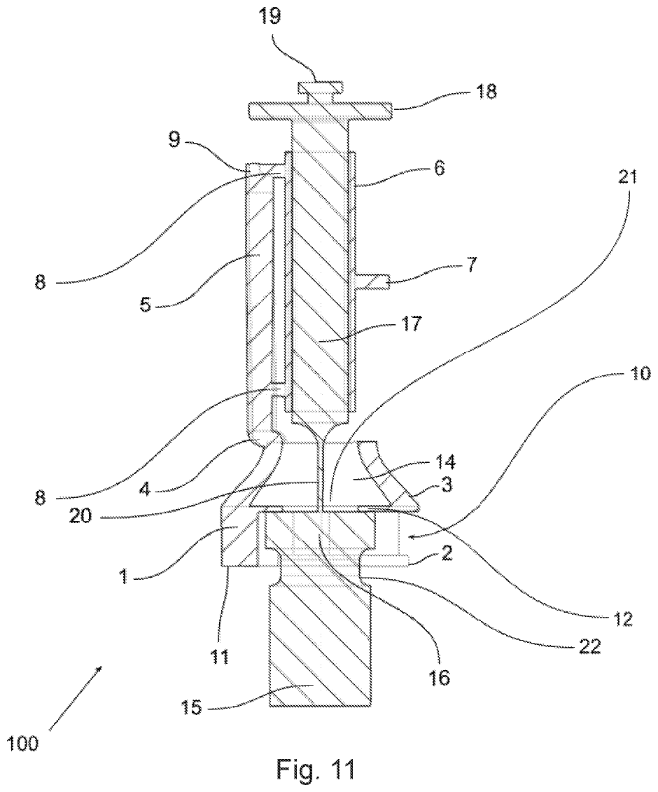

The vial body 15 can be inserted into the vial opening 10, as seen in FIG. 11, such that the vial cap 16 is inside the sheath 1, and the upper surface of the vial cap 16 is resting on, or near, a stabilizing contact surface 12. The lower surface of the vial cap 16 can slide and rest on front face protrusions 2 (as shown in FIG. 11) such that the vial body 15 cannot become disconnected from the device 100 via the opening in 11, and can only be removed from the device 100 by withdrawal of the vial cap 16 via the vial opening 10. If the vertical height of the vial cap 16 and the height of the vial opening 10 are substantially the same, the vial cap 16 would contact both the front face protrusion 2 and stabilizing contact surface 12.

The syringe barrel 17 can be operably connected to the clamp 6. While the device 100 is designed such that syringe barrel 17 is pressed into the clamp 6, temporarily deforming the clamp 6, and then being maintained within the clamp 6 via a friction fit, the operably connection between syringe barrel 17 and the clamp 6 is not limited to a friction fit, and can include other connection modalities as well, including an adhesion substance.

The syringe barrel 17 can move axially/vertically along an interior surface of the clamp 6 by applying pressure to the syringe barrel 17, handle 7, rail connector 5 and/or rail connector end 9. The syringe barrel 17 is maintained at the shifted position by the radial friction and/or force of the portion of the clamp 6 that contacts the syringe barrel 17.

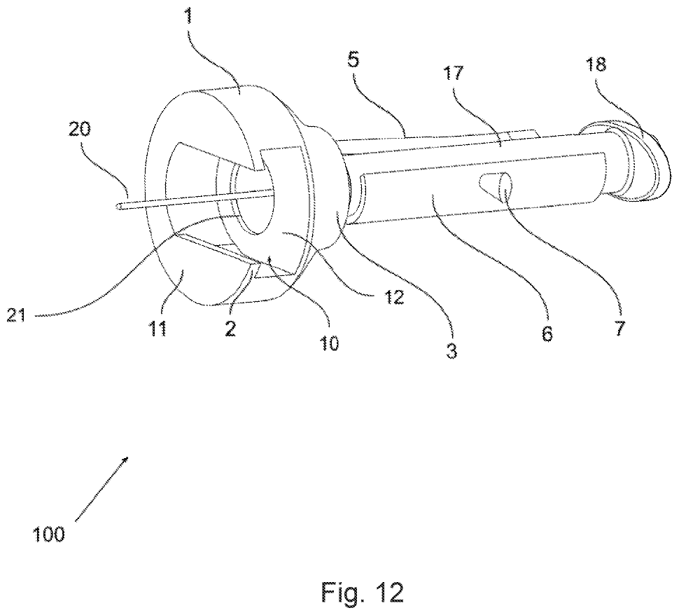

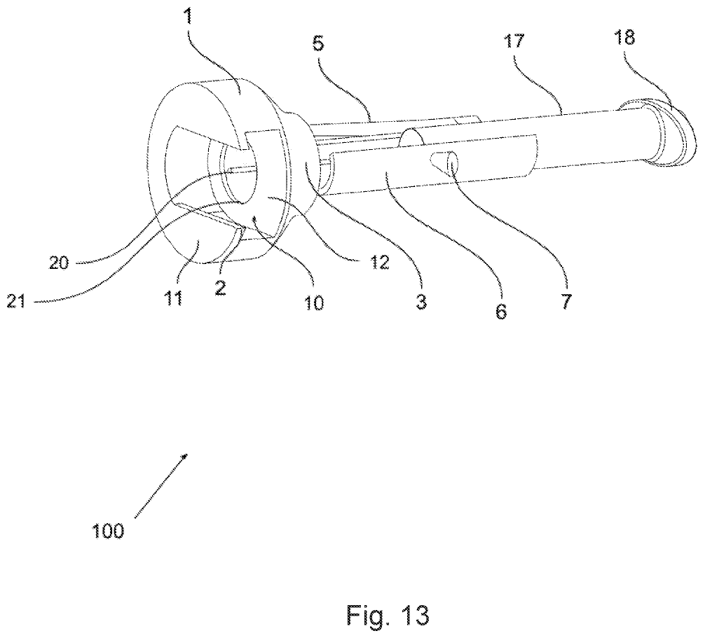

As seen in FIG. 12, the uppermost surface of the clamp 6 can contact the syringe tab 18 if the syringe barrel 17 is in the lowest position. As can be seen in FIG. 13, if the syringe barrel 17 is in its position as compared to FIG. 12, the syringe needle 20 is surrounded radially by the sheath 1, and the syringe needle 20 is vertically closer to the clamp 6 than the front face 11 of the sheath 1.

As can be seen in FIG. 11, upon placement of the syringe barrel 17 within the clamp 6, a syringe tab 18 and a syringe plunger 19 remain accessibly to the user and the device 100 does not obstruct operation of the syringe plunger 19, and allows for viewing of the measurement lines on the syringe barrel 17, so that a user can view and know the dose stored within the syringe barrel 17, as well as an administered dose. Additionally, a syringe needle 20 is separated from a user by the interposition of either through the sheath 1, an inner tapered neck 14, or the clamp 6.

The structure of device 100 and syringe barrel 17 to move relative to each other, can maintain separation between the syringe needle 20 and a user, through a whole range of motion and use of the syringe barrel 17.

The structural configuration of the device 100 causes the syringe needle 20, the backing hole 21, and the vial cap 16 to all be substantially concentric, allowing for insertion of said syringe needle 20 into the vial for filling.

The structural configuration of the device 100 is such that the syringe needle 20 is encompassed in the inner tapered neck 14 (and/or the sheath 1) when not being used during withdrawal or injection.

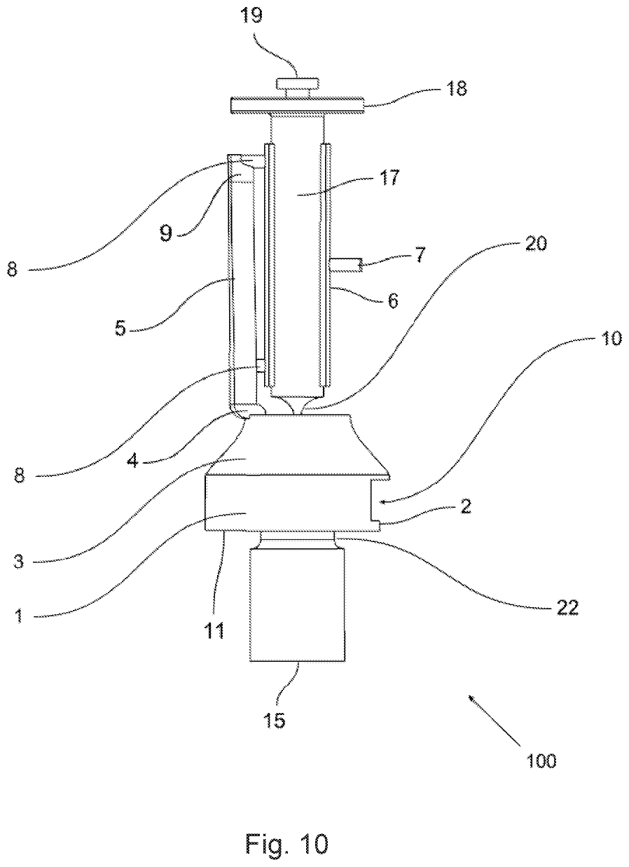

The orientation of the device 100 as shown in FIGS. 9 and 10 demonstrates a first step of an injection process, where the syringe barrel 17 is connected to the clamp 6, and the vial body 15 is connected to the device 100 via the vial opening 10. The front facing protrusions 2 make the vial opening 10 smaller, which is configured to allow the vial neck 22 to slide into place, but does not allow the vial cap 16 to be removed axially. The device 100 is moved via the handle 7 such that the syringe needle 20 goes through the backing hole 21, and into the interior of the vial body 15. The handle 7 is shown in one location in the figures for exemplary purposes, but in other embodiments the handle 7 include two or more handles, and can be on any other suitable location of the clamp 6. Additionally, the handle 7 can be solid or hollow, and can have any suitable cross-sectional shape extending from the between the clamp 6, including but not limited to a substantially rectangular shape, a substantially conical shape, a substantially cylindrical shape, etc. From this configuration, the substance (liquid and/or aqueous solution) present in the vial can be withdrawn by pulling the syringe plunger 19. After a sufficient amount of the substance from the vial body 15 has been withdrawn into the syringe barrel 17, the syringe barrel 17 and device 100 can be moved such that the syringe needle 20 exits the vial body 15, while still being separated from the user by the sheath 1 and/or the tapered neck 3. Once the substance is sufficiently removed from the vial body 15, the vial body 15 can be removed from the device 100 through the vial opening 10.

Upon removal of the vial body 15 from the device 100, the user can perform an injection with the syringe barrel 17 in contact with the clamp 6, such that the skin of a patient contacts the front face 11. Once the front face 11 is positioned to contact the skin of the patient, the syringe can be moved by pushing on the syringe tab 18 such that the syringe needle 20 moves through the backing hole 21 and is inserted into the patient. Then, the substance within the syringe barrel 17 can be administered to the patient by pressing the syringe plunger 19.

Once the substance is administered, the entire device 100, including the syringe needle 20, can be removed from the patient by pulling from the syringe tab 18. During this pulling process, the device 100 will remain connected to the syringe barrel 17 through syringe barrel 17's contact with the clamp 6. After removal of the syringe needle 20 from the patient, and after movement of the syringe barrel 17 such that the syringe needle 20 is separated from a user (and separated from the patient) by the interposition of the inner tapered neck 14 and/or the sheath 1, the syringe barrel 17 and the device 100 can be disposed of in the configuration shown in FIG. 13. Alternatively, the syringe barrel 17 can be completely removed from the device 100 and the syringe barrel and/or the device 100 can then be disposed of.

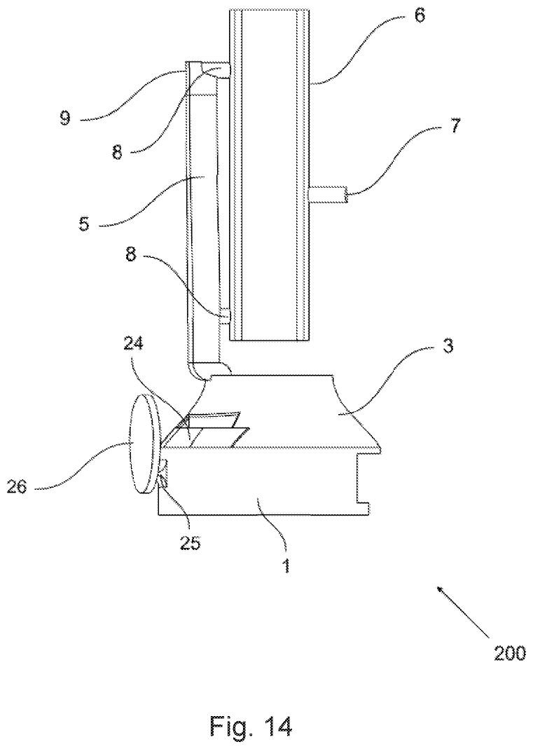

A second embodiment of a device 200 is shown in FIGS. 14-20. In the second embodiment of the device 200, the same reference numbers refer to similar elements as compared to device 100 and operation of the device 200 is similar other than the differences noted below.

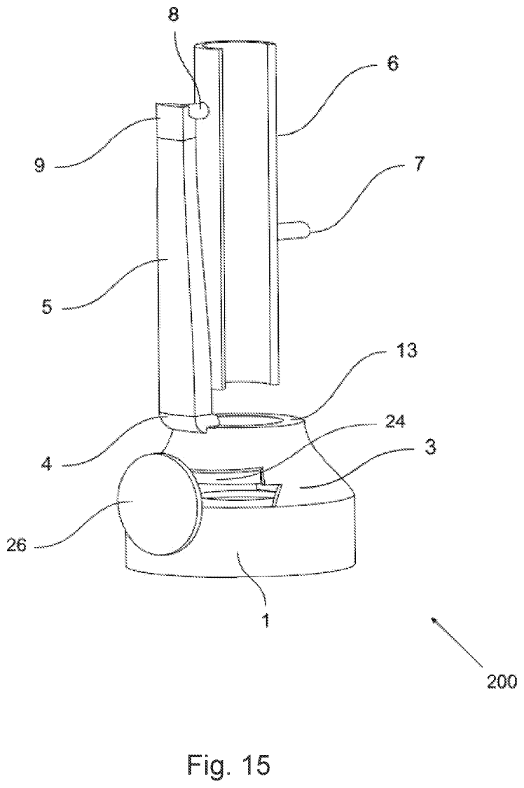

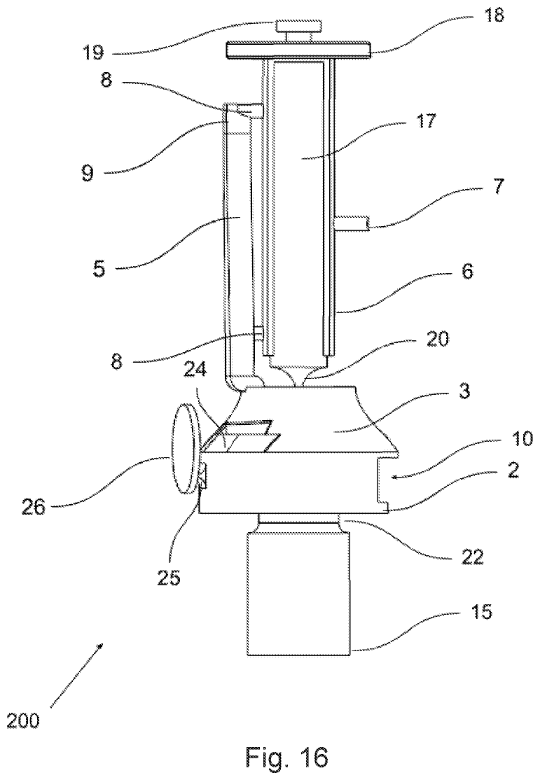

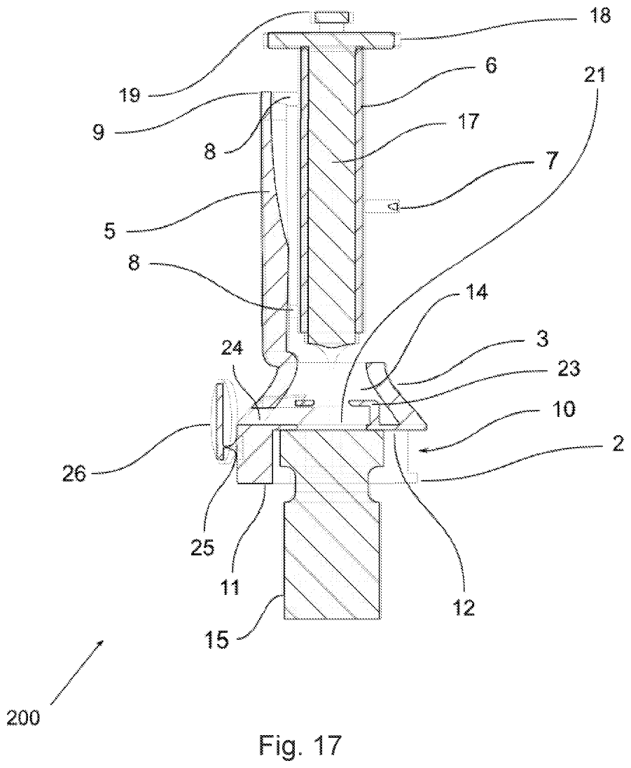

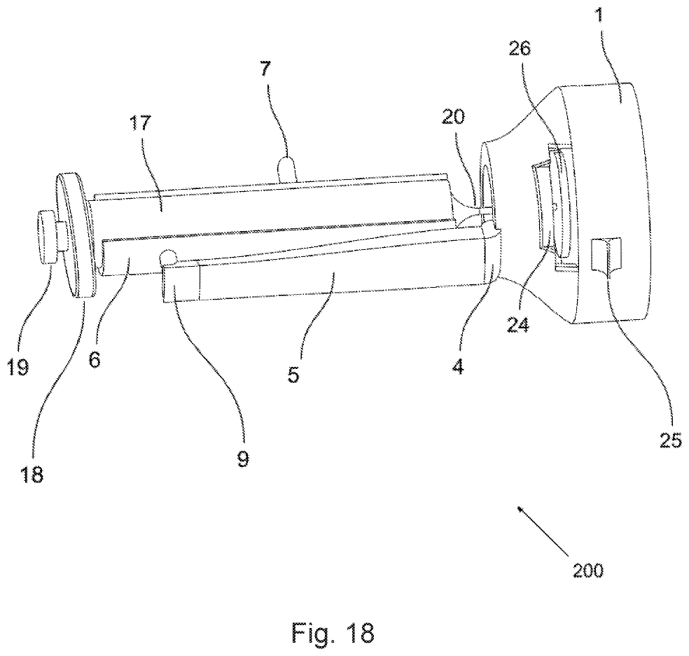

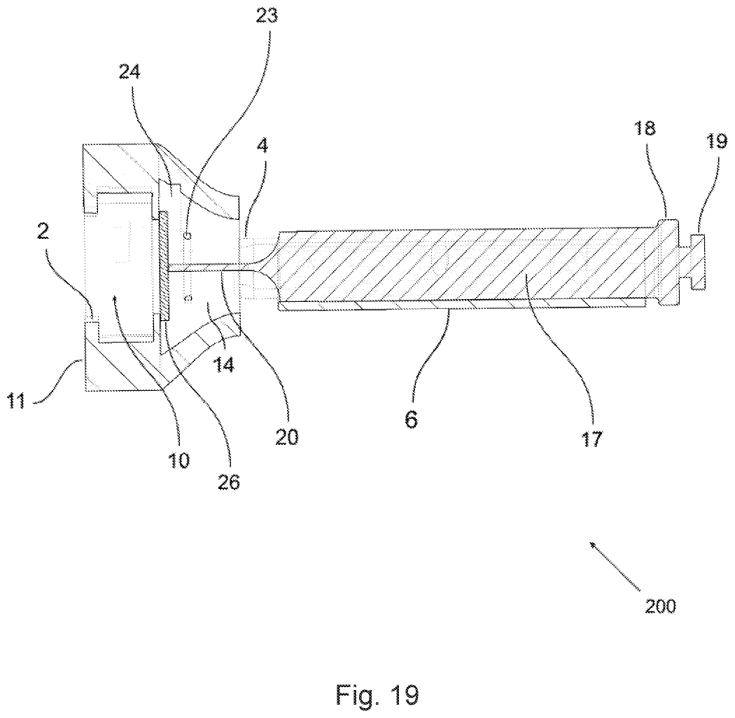

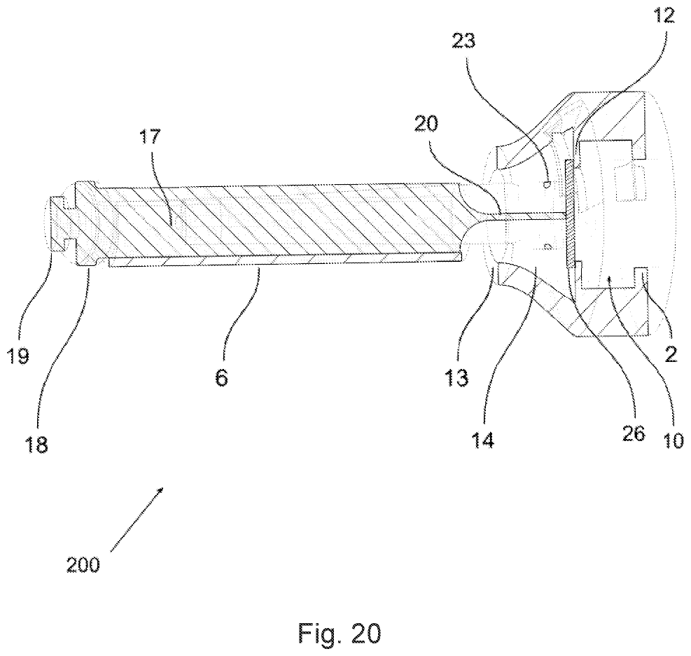

FIG. 14 shows a top perspective view of a second embodiment of the device, including a stopper. FIG. 15 shows a left perspective view of the device of FIG. 14. FIG. 16 shows a top perspective view of the second embodiment of the device, with the stopper component visible, attached to a conventional syringe with a vial loaded into the vial stabilizer. FIG. 17 shows a top side cross sectional view of the assembled devices of FIG. 16. FIG. 18 shows a left perspective view of the second embodiment of the device with the stopper in place after being inserted into the slot. The device is attached to a conventional syringe and the needle is in contact with the stopper, with the vial stabilizer unloaded. FIG. 19 shows a right cross sectional view of the device of FIG. 18. FIG. 20 shows a left cross sectional view of the device of FIG. 18.

As can be seen in FIGS. 14-20, embodiment 200 is similar to embodiment 100, but embodiment 200 includes a stopper 26, a stopper mount 25, a slot 24 and a backing plate 23. The stopper 26 is configured for placement in the slot 24 after an injection with the device 200, and creates a physical material barrier between the syringe needle 20 and a user external to the device 200. Thus, even if the syringe barrel 17 receives a vertical/axial force towards the sheath 1, the syringe needle 20 will contact the stopper 26.

In FIGS. 14-17, the stopper 26 is operably connected to the device 200 by the stopper mount 25. In some embodiments, such as those shown in FIGS. 18-20, the stopper mount 25 is formed of a material that is breakable through a relatively low level of pressure applied by a user's hand on the stopper 26, thus separating the stopper 26 from the device 200. In other embodiments, the stopper mount 25 is formed of a flexible material or a material of sufficient length such that the stopper 26 can be inserted into the slot 24 without breaking the stopper mount 25.

Upon movement of the stopper 26, the stopper 26 is inserted through the slot 24 until the stopper 26 contacts and/or rests on the backing plate 23 and/or the stopper 26 contacts and/or rests on the contact surface 12 around the backing hole 21, as shown in FIGS. 18-20.

The backing plate 23 is configured to extend from a portion of the contact surface 12 a distance vertically away from the front face protrusion 2. The backing plate 23 can also extend around a portion of the perimeter of backing hole 21, with the backing plate 23 configured to maintain the stopper 26 in a fixed position relative to the device 200 upon insertion of the stopper 26 into the slot 24.

The device 200 can remain in the configuration shown in FIGS. 18-20 through disposal of the device 200, such that the stopper 26 remains as a physical barrier between a user and the syringe needle 20.

Each embodiment of the device 100 and the device 200 discussed above may be formed from any suitable material. For example, each element of the needlestick protection device can be formed of the same material, or be formed of different materials. Specifically, each element of the device 100 and the device 200 can be formed of one or more of the following material examples, including, but not limited to: any suitable plastic(s), such as Polycarbonate (PC); Polymethyl Methacrylate (PMMA); Polyethylene Terephthalate (PET); Polyvinyl Chloride (PVC); Polypropylene (PP); Polystyrene (PS); Polycarbonate (PC); Polyamide; Acrylonitrile butadiene styrene (ABS); Polyethylene; Thermoplastic Polyurethane (TPU); Styrene Methyl Methacrylate (SMMA); Amorphous Copolyester (PETG); Fluorinated Ethylene Propylene (FEP): and/or any suitable rubber(s), olefin(s), and/or resin(s), such as Liquid Silicone Rubber (LSR); Cyclic Olefin Copolymers; Ionomer Resin; Styrene Acrylonitrile Resin (SAN); Polystyrene (General Purpose— GPPS): and/or any suitable glass material: and/or any metal(s): and/or any suitable carbon based materials such as graphene and carbon fiber.

Elements disclosed as being “operably attached” or “operably connected” can refer to any suitable attachment, such as by adhesives and/or mechanical attachment. Additionally, elements disclosed as being “operably attached” or “operably connected” can refer to those elements being formed as a unitary element.

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

### PARTS LIST

1. Sheath
2. Front face protrusions
3. Tapered neck
4. Connector region
5. Connector rod
6. Clamp
7. Handle
8. Connector posts
9. Connector rail end
10. Vial opening
11. Front face
12. Contact surface
13. Tapered backing
14. Inner tapered neck
15. Vial Body
16. Vial Cap
17. Syringe Barrel
18. Syringe Tab
19. Syringe Plunger
20. Syringe Needle
21. Backing hole
22. Vial neck
23. Backing plate for stopper
24. Slot
25. Stopper mount
26. Stopper

What is claimed is:

1. A device, comprising:
a sheath, wherein the sheath comprises an opening configured to receive a container;
a connector conduit, a first end of the connector conduit operably connected to the sheath,
a connector rod, wherein a second end of the connector conduit, the second end of the connector conduit opposite the first end of the connector conduit, is operably connected to a first end of the connector rod; and
a clamp, wherein a second end of the connector rod, the second end of the connector rod opposite the first end of the connector rod, is operably connected to the clamp, and wherein the clamp is configured to extend at least partially around the perimeter of a syringe barrel.

2. The device of claim 1, wherein the connector conduit is operably connected to the first end of the connector conduit.

3. The device of claim 1, wherein the sheath comprises a substantially flat face on one surface of the sheath, wherein the substantially flat surface is configured to contact a patient.

4. The device of claim 1, wherein the connector region tapers from a first diameter to a second diameter, the second diameter being smaller than the first diameter.

5. The device of claim 4, wherein the first diameter is a diameter of the sheath.

6. The device of claim 1, further comprising a front face protrusion and a contact surface, wherein the front face protrusion and the contact surface are configured to maintain a position of a container relative to the sheath.

7. The device of claim 1, wherein the clamp is configured to substantially secure a syringe barrel in two or more locations, with one of the two or more vertical/axial locations being nearer the sheath than another of the two or more vertical/axiallocations.

8. The device of claim 7, wherein the clamp substantially secures the syringe barrel through a friction force between the syringe barrel and the portion of the clamp that contacts the syringe barrel.

9. The device of claim 1, further comprising a tab configured to receive pressure from a user.

10. The device of claim 1, further comprising a slot in the tapered neck, wherein the slot is configured to hold a stopper.

11. The device of claim 10, wherein the stopper is configured to rest within the slot between a backing hole of the device and a lower surface of the clamp and/or the stopper backing plate.

12. A method of preparing an injection, comprising:
moving a vial into an opening of a device, the vial comprising a container, a vial cap and a liquid and/or aqueous solution, the device comprising:
a sheath, wherein the sheath comprises an opening configured to receive a container;
a connector conduit, a first end of the connector conduit operably connected to the sheath,
a connector rod, wherein a second end of the connector conduit, the second end of the connector conduit opposite the first end of the connector conduit, is operably connected to a first end of the connector rod; and
a clamp, wherein a second end of the connector rod, the second end of the connector rod opposite the first end of the connector rod, is operably connected to the clamp, and wherein the clamp is configured to extend at least partially around the perimeter of a syringe barrel of a syringe;
inserting the syringe barrel into the clamp;
inserting a needle of the syringe into the vial;
withdrawing at least a portion of the liquid and/or aqueous solution through the needle, into the syringe barrel;
removing the needle from the vial; and
removing the vial from the opening of the device.

13. The method of claim 12, further comprising, after the step of removing the vial from the opening of the device, the steps of
contacting a patient's skin with a contact surface of the device, sliding syringe barrel along clamp so that the needle enters a portion of the patient, and
applying pressure to a syringe plunger of the syringe.

14. The method of claim 12, wherein the device further comprises a front face protrusion and a contact surface, and wherein a vial cap of the vial rests on the contact surface.

15. The method of claim 12, wherein the device further comprises a slot in the tapered neck, wherein the slot is configured to hold a stopper, and wherein the method further comprises, after the step of removing the vial from the opening of the device, moving the stopper into the slot.
